(19) United States
(12) Patent Application Publication
(10) Pub. No.: US 2003/0087864 A1
(43) Pub. Date: May 8, 2003

(54) DNA vaccine against staphylococcus aureus

(76) Inventors: Brian Talbot, Lennoxville (CA); Eric Brouillette, Sherbrooke (CA); Pierre Lacasse, Lennoxville (CA)

Correspondence Address: MERCHANT & GOULD PC, P.O. BOX 2903, MINNEAPOLIS, MN, 55402-0903, US

(21) Appl. No.: 10/193,577

(22) Filed: Jul. 9, 2002

(30) Foreign Application Priority Data
Jul. 9, 2001 (CA) 2,351,018

(51) Int. Cl. A61K 48/00

(52) U.S. Cl. 514/44

(57) ABSTRACT

The present invention relates to the use of a plasmid encoding Staphylococcus aureus polypeptides and its use in the preparation of compositions and vaccines. More specifically, the present invention is concerned with compositions, DNA vaccines and methods for providing an immune response and/or a protective immunity into mammals against a Staphylococcus aureus associated disease, such as mastitis. The plasmid used in the composition or DNA vaccine comprises at least one nucleotide coding sequence of a Staphylococcus aureus polypeptide, such as the Clumping factor A (ClfA), the fibronectin-binding protein A, the sortase-A or the pre-pheromone (ArgD).

### FIELD OF THE INVENTION

The present invention relates to compositions, DNA vaccines and methods for providing an immune response and/or a protective immunity into mammals, particularly humans and bovines, against a Staphylococcus aureus associated disease.

### BACKGROUND OF THE INVENTION

Staphylococcus aureus is a potentially pathogenic bacteria found in nasal, skin, hair follicles, and perineum of warm-blooded mammals, such as human and bovines. This bacteria may cause a wide range of infections and intoxications. Recently, Staphylococcus aureus has been identified as the most important causative organism of bovine mastitis.

Mastitis is one of the most important and costly diseases of dairy cow herds. It is found in 19 to 45% of cattle during lactation worldwide. Despite treatment and different levels of infection, mastitis has long-lasting effects on the milk yield of infected animals. Bovine mastitis has also become an important environmental issue because of increasing public resistance to the use of antibiotics and the development of resistance strains of the pathogens.

Staphylococcus aureus vaccines are presently available in the form of inactivated highly encapsulated S. aureus cells. Their efficiency for long-term treatment of mastitis has not been confirmed. Several attempts have been made to formulate vaccines against S. aureus using capsular polysaccharide alone or in combination with staphylococcus alpha toxin. There is however considerable variability in the structure of capsular polysaccharides which could limit the usefulness of this approach. Various recombinant adhesion proteins have also been used with some success either alone or in combination with non toxic epitopes of alphatoxin and purified capsular polysaccharide. Recent progress has been made with the use immunogens such as poly-N-succinyl-beta 1,6, glucosan which appear to be produced only in vivo. This approach has been successful in inducing a protective response against kidney infection by S. aureus in the rat. Similarly it has recently been demonstrated that mice could be protected after immunization with proteins or peptides involved in the regulation of expression of extracellular S. aureus proteins. There has only been one report demonstrating that DNA immunization may be used for protection against S. aureus infection by vaccinating mice with DNA containing the mecA gene of S. aureus encoding for the penicillin-binding protein PBP2′ (OHWADA, A., M. et al. 1999. DNA vaccination by mecA sequence evokes an antibacterial immune response against methicillin-resistant Staphylococcus aureus. J. Antimicrob. Chemother. 44:767-774). Studies of Staphylococcus aureus vaccines against bovine mastitis have been limited mostly to the bacterin or capsular polysaccharide forms. No-one has used in vaccines S. aureus polypeptides such as adhesion proteins, extracellular regulatory proteins, and autoinducing peptides for inducing an immune response against S. aureus, and especially for preventing and/or treating mastitis.

Therefore, there is a need for new Staphylococcus aureus vaccines which target_ new proteins and more particularly to compositions and DNA vaccines that comprise a plasmid which includes at least one nucleotide coding sequence of a Staphylococcus aureus polypeptide selected from the group consisting of adhesion proteins, extracellular regulatory proteins, and autoinducing peptides for expressing the polypeptide in a mammal.. There is also a need for new methods for the prevention or treatment of S. aureus associated diseases, such as mastitis.

### SUMMARY OF THE INVENTION

An object of the invention is to provide compositions, DNA vaccines for their use in the elicitation of an immune response or a protective immunity against Staphyloccocus aureus in a mammal or for their use in the treatment or prevention of Staphyloccocus aureus associated diseases.

According to an aspect of the invention, the composition and the DNA vaccine comprises a plasmid and a pharmaceutically acceptable carrier. The plasmid comprises at least one nucleotide coding sequence of a Staphylococcus aureus polypeptide selected from the group consisting of adhesion proteins, extracellular regulatory proteins, and autoinducing peptides. The plasmid further comprises transcriptional and translational regulatory sequences operably linked to the nucleotide coding sequence for expressing the polypeptide in a mammal.

According to another aspect of the invention, there is provided a method for eliciting an immune response against Staphyloccocus aureus in a mammal, the method comprising the step of administrating to the mammal an effective amount of a composition as defined above.

According to a further aspect of the invention, there is provided a method for eliciting a protective immunity against a Staphyloccocus aureus associated disease in a mammal, the method comprising administering to the mammal an effective amount of a composition as defined above.

Yet, according to another aspect of the invention, there is provided a method for preventing and/or treating a Staphyloccocus aureus associated disease in a mammal, comprising the step of administering to the mammal an effective amount of a DNA vaccine as defined above.

According to another aspect, the present invention proposes the use of a composition as defined above or a DNA vaccine as defined above, for eliciting an immune response against Staphyloccocus aureus in a mammal, or for eliciting a protective immunity against a Staphyloccocus aureus infection in a mammal

According to a further aspect, the present invention proposes the use of a composition as defined above or a DNA vaccine as defined above for preventing and/or treating a Staphyloccocus aureus associated disease in a mammal.

According to a preferred embodiment, the compositions, DNA vaccines and methods of the invention are useful for treating and/or preventing a Staphyloccocus aureus associated disease, such as pneumonia, mastitis, phlebitis, meningitis, and urinary tract infections, osteomyelitis and endocarditis, but most preferably mastitis.

An advantage of the present invention is that it provides compositions and DNA vaccines that target proteins not used in marketed vaccines. Furthermore, the compositions and DNA vaccines of the present invention aim at eliciting an immune response to block important virulence factors of S. aureus that have key roles during each phase of infection and disease development, and particularly in the case of the mastitis pathogenesis.

### BRIEF DESCRIPTION OF THE FIGURES

FIG. 1 is a schematic representation of nucleotide sequences encoding S. aureus polypeptides included in a plasmid vector of a composition according to a preferred embodiment of the invention.

FIG. 2 are pictures illustrating the expression of the S. aureus proteins encoded by the plasmid vectors of FIG. 1. The first row of pictures shows transfected COS-1 cells with the plasmid indicated above.

FIG. 3 is a picture of a gel which illustrates the secretion and N-glycosylation analysis of the fusion protein encoded by the pCTLA-ClfA. COS-1 cells were transfected with pClfA or pCTLA-ClfA in the absence or presence of tunicamycin to inhibit N-glycosylation: (1) pClfA, (2) pCTLA-ClfA and (3) pCTLA-ClfA+tunicamycin.

FIG. 4 are graphs showing antibody assay results with sera from DNA immunized BALB/c mice. (A) ELISA for total IgG and (B) IgG isotypes are shown as mean O.D. values from six mice with bars indicating standard deviation. (C) Sera from pClfA immunized mice were used to test the ability of antibodies to recognize_ the native ClfA on the surface of bacteria. Mean values and standard deviation from triplicates are shown. Groups where the recombinant protein was used for the second boost are indicated (prt).

FIG. 5 are graphs showing proliferative response of splenocytes from DNA vaccinated mice after in vitro stimulation with recombinant ClfA(221-550). Stimulation index (SI) is the CPM ratio of stimulated cells to unstimulated cells. Mean values of SI and standard deviation for quadruplicates are shown. Statistical significance versus proliferation of splenocytes from pCI vaccinated mice is indicated. **P<0.01 *P<0.05.

FIG. 6 is a bar graph showing the inhibition results of S. aureus binding to fibrinogen by sera from DNA vaccinated mice. Main values are shown with bars indicating standard deviation for triplicates. *P<0.005.

FIG. 7 is a bar graph showing the In vitro phagocytosis results of S. aureus after opsonisation. Mean values for triplicates are shown with bars indicating standard deviation. Statistical significance of sera from pClfA vaccinated mice versus the pCI vaccinated one is indicated. **P<0.001 *P<0.01.

FIG. 8 is a graph showing the in vivo opsonisation of S. aureus results in DNA vaccinated mice.

FIG. 9 is a graph showing the production of specific IgG1 antibodies in mice following administration of DNA vaccines according to a preferred embodiment of the invention, and more particularly DNA vaccines comprising a bicistronic plasmid vector. The letters represent the nucleotide encoding sequence or combination of nucleotide coding sequence expressed by the plasmid. S=sortase, A=AIP, D=D1D3 of fibronectin binding protein, Clfa=Clumping factor A, D-Clfa=a single plasmid expressing both D1D3 and Clfa, but fused in the plasmid in the order D1D3 followed by Clfa, antibody against D1D3 was measured; the second D-Clfa is the same serum but tested against Clfa; C-S=a single plasmid expressing both Clfa and sortase, antibody against sortase; C-A=a single plasmid expressing both Clfa and AIP, antibody against AIP; C-D=a single plasmid expressing both ClfA and DID3 but in the order Clfa followed by D1D3, antibody measured against D1D3.

### DETAILLED DESCRIPTION OF THE INVENTION

The present invention is directed to the use of a plasmid encoding Staphylococcus aureus polypeptides and its use in the preparation of compositions and vaccines. More specifically, the present invention is concerned with compositions, DNA vaccines and methods for providing an immune response and/or a protective immunity into mammals against a Staphylococcus aureus associated disease.

As used herein, the term “immune response” refers to a cytotoxic T cells response or increased serum levels of antibodies to an antigen, or to the presence of neutralizing antibodies to an antigen, such as a S. aureus polypeptide. The term “protection” or “protective immunity” refers herein to the ability of the serum antibodies and cytotoxic T cell response induced during immunization to protect (partially or totally) against disease caused by an infectious agent, such as a S. aureus. That is, a mammal immunized by the compositions or DNA vaccines of the invention will experience limited growth and spread of an infectious S. aureus.

A non-exhaustive list of S. aureus associated diseases against which the methods, compositions and DNA vaccines of the invention may be useful, includes pneumonia, mastitis, phlebitis, meningitis, and urinary tract infections, osteomyelitis and endocarditis. Among the previous mentioned disease, mastitis is preferred.

1. Plasmid

The plasmid contemplated by the present invention comprises at least one nucleotide coding sequence of a S. aureus polypeptide selected from the group consisting of the adhesion proteins, extracellular regulatory proteins, and auto inducing peptides (AIP). The plasmid further comprises transcriptional and translational regulatory sequences operably linked to the nucleotide coding sequences for expressing the polypeptide in a mammal. By the term “transcriptional and translational regulatory sequences” is meant nucleotide sequences positioned adjacent to a DNA coding sequence which direct transcription or translation of a coding sequence (i.e. facilitate the production of, e.g., Clfa, FnBp, or sortase protein). The regulatory nucleotide sequences include any promoter sequences which promote sufficient expression of a desired coding sequence (such as Clfa, FnBp, or sortase) and presentation of the protein product to the inoculated mammal's immune system such that an immune response and/or a protective immunity is provided. The “promoter sequence” may consist of a minimal sequence of nonretroviral or retroviral origin sufficient to direct transcription. Any promoter or combination of promoters suitable for cloning and expression of the S. aureus nucleotide sequence may be used in accordance with the present invention. More specifically, the promoter may be selected, without limitation, from the group consisting of the cytomegalovirus immediate-early enhancer promoter and the PC2 (proprotein convertase 2). The enhancer sequence may or may not be contiguous with the promoter sequence. Enhancer sequences influence promoter-dependent gene expression and may be located in the 5′ or 3′ regions of the native gene. More specifically, the enhancer sequences may be selected without limitation, from the group consisting of HCMV EI, HTLV-1 Enhancer promoter, Rous sarcoma virus enhancer-promoter, parvovirus P6 enhancer, SV40 enhancer, IgH 3′ enhancer. Expression is constitutive or inducible by external signals or agents. Optionally, expression is cell-type specific, tissue-specific, or species specific.

By the term “operably linked to transcriptional and translational regulatory sequences” is meant that a polypeptide coding sequence and minimal transcriptional and translational controlling sequences are connected in such a way as to permit polypeptide expression when the appropriate molecules (e.g., transcriptional activator proteins) are bound to the regulatory sequence(s). In the present invention, polypeptide expression in a target mammal cell is particularly preferred.

A non-exhaustive list of adhesion proteins that are used according to the present invention includes Clumping factor A (ClfA), fibronectin-binding protein A and B (FnBp-A and FnBp-B), collagen binding protein (Cna), and fibrinogen-binding protein (EFB). Preferred adhesion proteins contemplated by the present invention are ClfA and FnBp-A or their functional derivative. A preferred extracellular regulatory protein consists of a sortase-A or its functional derivative whereas a preferred autoinducing peptide is the pre-pheromone (ArgD) or its functional derivative. As used herein, the term “functional derivative” refers to a protein/peptide sequence that possesses a functional immunological activity that is substantially similar to the immunological activity of the whole protein/peptide sequence, i.e. it elicits an immune response of a protective immunity against S. aureus. A functional derivative of a protein/peptide may or may not contain post-translational modifications such as covalently linked carbohydrate, if such modification is not necessary for the performance of a specific function. The term “functional derivative” is intended to the “fragments”, “segments”, “variants”, “allelic variants”, “analogs” or “chemical derivatives” of a protein/peptide.

According to a preferred embodiment, the nucleotide coding sequence concerning the clumping factor A comprises nucleotides 1 to 3499, and more specifically nucleotides 962 to 1951 of GenBank accession no. Z18852. According to another preferred embodiment, the nucleotide coding sequence concerning fibronectin-binding protein A comprises nucleotides 1 to 3342, and more specifically nucleotides 962 to 1951 and/or nucleotides 2538 to 2578 of GenBank accession no. J04151. According to a further preferred embodiment, the nucleotide coding sequence concerning sortase-A comprises nucleotides 1 to 1256, and more specifically nucleotides 443 to 1147 of GenBank accession no. AF162687. Yet, according to another preferred embodiment, the nucleotide coding sequence concerning the pre-pheromone comprises nucleotides 1 to 1691, and more specifically nucleotides 158 to 180 of GenBank accession no. AF026120.

Advantageously, a preferred plasmid contemplated by the present invention is a bicistronic plasmid. As used herein, the term “bicistronic plasmid ” refers to a plasmid that comprises two nucleotide encoding sequences that each encodes for a S. aureus polypeptide as previously described. In this connection, a preferred bicistronic plasmid comprises a nucleotide encoding sequence of the clumping factor A and a nucleotide encoding sequence of either the fibronectin-binding protein A, the sortase-A or the pre-pheromone.

The nucleotide coding sequence may also be linked to any coding sequences that improves the immune response to the antigen, i.e. the encoded S. aureus polypeptide. Preferred coding sequence include any suitable antigen presenting cell targeting sequences, such as cytotoxic lymphocyte T antigen 4 (CTLA4) sequence; integrated CpG sequences; cytokine_ coding sequences, such as granulocyte macrophage colony stimulator factor (GM-CSF); internal ribosome entry site (IRES) sequences and secretion signal sequences. According to a preferred embodiment, the nucleotide coding sequence is linked to the CTLA-4 sequence which consists of nucleotides 1 to 666, and most preferably nucleotide 1 to 476 of GenBank accession no. X93305. Most preferably, the nucleotide coding sequence linked to the CTLA-4 sequence may further be linked to the human IgG1 gene which comprises nucleotide 1 to 1827 of GenBank accession no. AF237583. Advantageously, the nucleotide coding sequence linked to the CTLA4 sequence are preferably linked to fragments of the human IgG1 gene which preferably relate to the hinge, CH2 and CH3 regions of the IgG1 gene which respectively consist of nucleotides 684 to 729, nucleotides 847 to 1177, and nucleotides 1275 to 1602 of GenBank accession no. AF237583.

Compositions and Vaccines

According to a first aspect, the present invention relates to a composition for eliciting an immune response or a protective immunity against S. aureus. According to a related aspect, the present invention relates to a DNA vaccine for preventing and/or treating a S. aureus associated disease. As used herein, the term “treating” refers to a process by which the symptoms of a S. aureus associated disease are ameliorated or completely eliminated. As used herein, the term “preventing” refers to a process by which a S. aureus associated disease are obstructed or delayed. The composition and the DNA vaccine of the invention comprises a plasmid as defined above and a pharmaceutically acceptable carrier.

As used herein, the term “pharmaceutically acceptable carrier” means a vehicle for containing the plasmid that can be injected into a mammalian host without adverse effects. Suitable pharmaceutically acceptable carriers known in the art include, but are not limited to, gold particles, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colors and the like.

Further agents can be added to the composition and vaccine of the invention. For instance, the composition of the invention may also comprise agents such as drugs, immunostimulants (such as α-interferon, β-interferon, γ-interferon, granulocyte macrophage colony stimulator factor (GM-CSF), macrophage colony stimulator factor (M-CSF), interleukin 2 (IL2), interleukin 12 (IL12), and CpG oligonucleotides), antioxidants, surfactants, flavoring agents, volatile oils, buffering agents, dispersants, propellants, and preservatives. For preparing such compositions, methods well known in the art may be used.

The amount of plasmid present in the compositions or in the DNA vaccines of the present invention is preferably a therapeutically effective amount. A therapeutically effective amount of plasmid is that amount necessary so that the nucleotide coding sequence of a S. aureus polypeptide performs its immunological role without causing, overly negative effects in the host to which the composition is administered. The exact amount of plasmid to be used and the composition/vaccine to be administered will vary according to factors such as the strength of the transcriptional and translational promoters used, the type of condition being treated, the mode of administration, as well as the other ingredients in the composition. Preferably, the composition or the vaccine formulation is composed of from about 10 μg to about 2 mg of plasmid.

For instance, during a mastitis vaccination program, bovines of about 8 months to several years old could be subjected to a (1 to 3) dose schedule of from about 50 μg to about 2000 μg of plasmid at 3 weeks afterward and 6 to 7 weeks afterward, and more preferably at 3, 6, 9 weeks. One or more of the plasmid used in this invention could be present in the composition or DNA vaccine from about 50 μg to about 2000 μg per dose.

2. Methods of Use

Another related aspect of the invention relates to methods for eliciting an immune response against Staphyloccocus aureus in a mammal by administrating to the mammal an effective amount of a composition as defined above, whereby expression of the nucleotide coding sequences in one or more cells in the mammal elicits a humoral immune response, a cell-mediated immune response, or both, against the S. aureus. As used herein, the term “mammal” refers preferably to a bovine, but may also refer to a human.

A further aspect of the invention relates to methods for eliciting a protective immunity against a Staphyloccocus aureus associated disease in a mammal by administering to the mammal an effective amount of a composition as defined above, whereby expression of the nucleotide sequences in one or more cells in the mammal elicits a humoral immune response, a cell-mediated immune response, or both against the S. aureus in the mammal, and whereby the mammal is protected from the disease caused by subsequent exposure to S. aureus.

The present invention is also concerned with delivery strategies or methods, such as priming the mammal to be protected with a composition or a DNA vaccine of the present invention followed by a boost with a suitable antigenic protein. Such an antigenic protein may be selected from the group consisting of the S. aureus adhesion proteins including Clfa, FnBp-A, collagen binding protein, fibrinogen-binding protein (EFB) and extracellular regulatory proteins such as sortase-A, and auto inducing peptides (AIP) such as the pre-pheromone (ArgD). In this connection, the preparation of such suitable antigenic proteins, any methods well known in the art may be used.

Yet, another aspect of the invention relates to a method for preventing or treating a S. aureus associated disease in a mammal. Therefore, the present invention specifically relates to methods which comprises the step of administering to the mammal in need thereof, an effective amount of a DNA vaccine as defined previously.

The DNA vaccine and the composition of the invention may be given to a mammal through various routes of administration. For instance, the composition may be administered in the form of sterile injectable preparations, such as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, intramuscularly or sub-cutaneously by injection or by infusion. The DNA vaccine and the composition of the invention may also be formulated as creams, ointments, lotions, gels, drops, suppositories, sprays, liquids or powders for topical administration. They may also be administered into the airways of a subject by way of a pressurized aerosol dispenser, a nasal sprayer, a nebulizer, a metered dose inhaler, a dry powder inhaler, or a capsule. Suitable dosages will vary, depending upon factors such as the amount of each of the components in the composition, the desired effect (fast or long term), the disease or disorder to be treated, the route of administration and the age and weight of the mammal to be treated. Anyhow, for administering the DNA vaccine and the composition of the invention, methods well known in the art may be used.

Bovines may be given, through various routes of administration a composition or a DNA vaccine according to the present invention. The efficiency of the DNA vaccine to induce a protective response against S. aureus mastitis in dairy cows, for instance, is preferably tested through challenge with S. aureus of mammary gland of vaccinated cows. The strain of the S. aureus preferably used for challenge of mammary gland is sensitive towards antibiotic treatments. A preferred challenge S. aureus strain contemplated by the present invention is S. aureus Newbould 305. Challenging of mammary gland of vaccinated cows, more specifically heifers, is preferably done after a significant immune response of heifers against S. aureus antigens encoded by the DNA vaccine of the present invention is evidenced in blood and milk samples and the absence of any natural occurring intra-mammary infections by a major pathogen. In this connection, the validation of such significant immune response, any methods well known in the art may be used.

### EXAMPLES

The following examples are illustrative of the wide range of applicability of the present invention and are not intended to limit its scope. Modifications and variations can be made therein without departing from the spirit and scope of the invention. Although any method and material similar or equivalent to those described herein may be used in practice for testing of the present invention, the preferred methods and materials are described.

### Example 1

### DNA Vaccines Directed Against ClfA and FnBP-A of S. aureus and Their Ability to Produce a Specific Immune Response

The present example shows that DNA vaccination against the fibrinogen-binding region of the clumping factor A allows the production of antibodies that bind to the surface of the bacteria, inhibit the adhesion to fibrinogen, promote the phagocytosis of S. aureus and promote the protection of mice following challenge with S. aureus.

### Materials and Methods

Bacteria

The bacteria used for the isolation of DNA was S. aureus 8325-4, generously donated by Dr. M. J. McGavin (Department of Microbiology, University of Toronto, Toronto, Canada). The adherence inhibition and phagocytosis tests were carried out with S. aureus Newman (ATCC 25904) since it is the reference strain for studies of clumping factor A and B. It is important to note that to better reflect real conditions neither of these two assays were carried out using a protein-A mutant. The recombinant proteins were produced with Escherichia coli BL21 (Promega Corp., Madison, USA) and the strain E. coli DH5α (Invitrogen Canada Inc., Burlington, Canada) was used for the DNA manipulations.

Production of Recombinant Proteins

Parts of FnbA and ClfA genes coding for the respective binding regions of FnBP-A and ClfA to ECM components were cloned into the pGEX-2T procaryotic expression vector (Amersham Pharmacia Biotech Inc, Baie d'Urfé, Canada). Genomic DNA from S. aureus was isolated by treatment with lysostaphin as previously described by Martineau, F., F. et al. (1998. Species-specific and ubiquitous-DNA-based assays for rapid identification of Staphylococcus aureus . J. Clin. Microbiol. 36:618-623). The D1-D3 portion of the fnbA gene that encodes the binding part of the FnBP-A to fibronectin was amplified by PCR, as described by Huff, S., Y. V. et al. (1994. Interaction of N-terminal fragments of fibronectin with synthetic and recombinant D motifs from its binding protein on Staphylococcus aureus studied using fluorescence anisotropy. J. Biol. Chem. 269:15563-15570). The DNA sequences of primers and oligonucleotides used in the present example are given in Table 1. The 380 bp product was cloned in pGEX-2T at the EcoR I and BamH I restriction sites. Similarly, a 1000 bp fragment from the portion A of the ClfA gene that is responsible for the binding to fibrinogen was amplified by PCR (McDevitt, D., P. et al. 1995. Identification of the ligand-binding domain of the surface-located fibrinogen receptor (clumping factor) of Staphylococcus aureus. Mol. Microbiol. 16:895-907) and cloned in the pGEX-2T at the same restriction sites. Note that a thymine (T) replaced a cytosine (C) at position 981 of ClfA gene from the S. aureus strain 83254 when the sequences of distinct cloned products were compared with the published sequence of S. aureus strain Newman (McDevitt, D., P. et al. 1995. Identification of the ligand-binding domain of the surface-located fibrinogen receptor (clumping factor) of Staphylococcus aureus. Mol. Microbiol. 16:895-907). This substitution caused the replacement of the amino acid valine by an alanine. Using E. coli BL21, the recombinant proteins were produced as fusion products with glutathione-S-transferase and called GST-D1-D3 and GST-ClfA(221-550), respectively. The proteins were affinity purified following instructions of the company (Amersham Pharmacia Biotech) including the addition of proteases inhibitors before lysis of the bacteria (2 μg/ml aprotinin, 2 mM EDTA and 100 μg/ml PMSF). When required, the recombinant part of the fusion proteins was cleaved from GST by digestion for 12 hours at RT with thrombin. After Coomassie staining, the protein D1-D3 demonstrated a single band by SDS-PAGE compared with one band and some degradation products for ClfA(221-550) as already reported (McDevitt, D., P. et al. 1995. Identification of the ligand-binding domain of the surface-located fibrinogen receptor (clumping factor) of Staphylococcus aureus. Mol. Microbiol. 16:895-907). The proteins were quantified by U.V. absorbance at 280 nm based on their amino acid composition using the following molecular extinction coefficients ε(M⁻¹ cm⁻¹): 44760, 3840, 74060 and 33140 for GST-D1-D3, D1-D3, GST-ClfA(221-550) and ClfA(221-550), respectively.

TABLE 1
Sequence of primers and oligonucleotides
used for the design of the plasmid vector of the present invention.

| | |
|---|---|
| Region | Sequence |

| | | | | |
|---|---|---|---|---|
| PCR Primers | D1-D3 | Sense | CCGGATCCGAAGGTGGCCAAAAT | (SEQ ID no.1) |
| | | Antisense | GCTCTAGATCATTCATTTTGGCCGCTT | (SEQ ID no.2) |
| | ClfA^{a} | Sense | CCGGATCCGTAGCTGCAGATGCACC | (SEQ ID no.3) |
| | | Antisense | GCTCTAGATCACTCATCAGGTTGTTCAGG | (SEQ ID no.4) |
| | CTLA-4^{b} | Sense | GGTCTAGAGGACCTCAGCACATTTGCC | (SEQ ID no.5) |
| | | Antisense | ATCCGGGCATGGTTCTGGATC | (SEQ ID no.6) |
| Oligonucleotides | Kosak | Sense | TCGAGCCACCATGG | (SEQ ID no.7) |
| | | Antisense | GATCCCATGGTGGC | (SEQ ID no.8) |
| | Hinge | Sense | TCTGGTGGCGGTGGCTCGGGCGGAGGTGGGTCGGGTGGCGGCG | (SEQ ID no.9) |
| | | Antisense | GATCCGCCGCCACCCGACCCACCTCCGCCCGAGCCACCGCCACCAGA | (SEQ ID no.10) |

^{a}the partial region of domain A coding for the binding regions was cloned
^{b}only the extramembranaire portion of the CTLA-4 gene was added in the plasmid

Production of Rabbit Antisera Against Recombinant Adhesins

The fusion proteins GST-D1-D3 and GST-ClfA(221-550) were injected subcutaneously (s.c.) in New Zealand white rabbits at a dose of 700 μg of protein in Freund's complete adjuvant. Booster injections with 150 μg of protein in incomplete Freund's adjuvant were carried out two weeks later. Sera were harvested 10 days after the booster injections.

Plasmid Constructions for Immunization of Mice

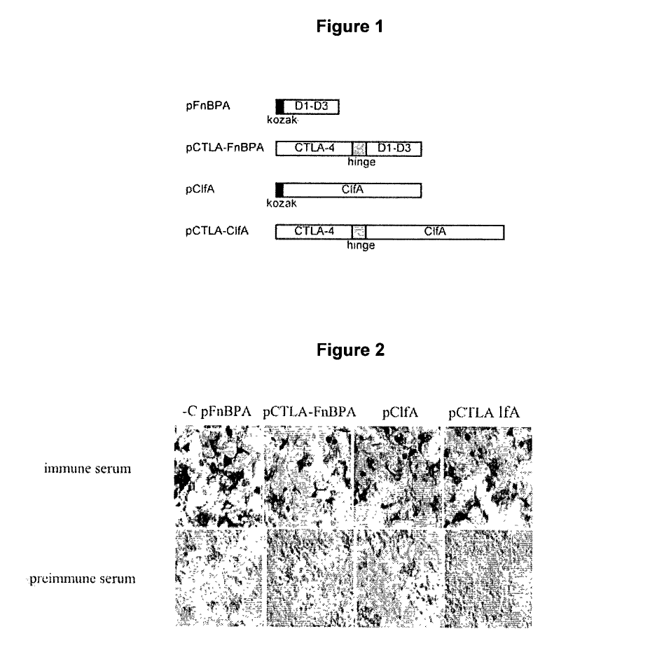

The DNA fragments coding for the binding regions of FnBP-A and ClfA were subcloned from the pGEX constructions to the pCI plasmid (Promega). The pCI is a plasmid with a cytomegalovirus promotor and SV40 intron for eucaryotic expression. In order to give an optimal mRNA translation, a short consensus sequence named <<Kozak>> was inserted in these first two pCI based constructions at the beginning of each coding sequence along with Xba I and BamH I sites. This sequence also contain the start codon. The new plasmids were called pFnBPA and pClfA. Two other plasmids were created from these constructions by replacing the Kozak sequence with the extracellular portion of the mouse cytotoxic lymphocyte T antigen 4 (CTLA-4). The extracellular portion of CTLA-4, that contains a secretion signal sequence, was amplified by PCR from a plasmid containing the cDNA of the gene and subcloned by Nhe I and BamH I in the pFnBPA and pClfA. Finally, using the BamH I site, a DNA sequence of 45 pb coding for a glycine and serine rich flexible region (hinge) was inserted between the extracellular portion of CTLA-4 and the adhesin binding regions of FnbA or ClfA gene. The two resulting plasmids were called pCTLA-FnBPA and pCTLA-ClfA. FIG. 1 shows the diagrams of the inserts for each construction. All the inserts were sequenced to confirm the integrity of the coding DNA (UCDNA services, University of Calgary, Calgary, Canada).

Expression of the Plasmids in Eucaryotic Cells

The ability of the plasmids to express the encoded antigen was tested by transfection into COS-1 cells grown in DMEM containing 10% fetal bovine serum (FBS) and 50 μg/ml of gentamicin. Lipofectamine (Invitrogen) was used for transfection according to the manufacturer instructions in 6-well culture plates. Immunohistochemistry techniques were applied to verify the expression of the antigens 48 hours after the transfection. Cells were fixed for 1 hour in 4% paraformaldhehyde and permeabilized with 50% methanol at −20° C. The wells were then saturated for 2 hours at RT with a blocking solution (Tris borate saline (TBS) pH 7.2, 2% milk powder, 2% bovine serum albumin, 0.05% saponin and 0.05% Tween). Rabbit immune sera against the recombinant GST fusion proteins (GST-D1-D3 or GST-ClfA(221-550)) were diluted 1/1000 and added for 2 hours at RT. Anti-rabbit Fab alkaline phosphatase conjugate (Sigma, St-Louis, USA) was used as secondary antibody at a dilution of 1/100. Between each step, 3 washes of 5 minutes were carried out using TBS-Tween 0.01%. The substrate solution contained 4-Nitroblue tetrazolium chloride (NBT) and X-phosphate (BCIP) (Roche diagnostics Inc., Laval, Canada) in 100 mM Tris (pH 9.5), 100 mM NaCl, 50 mM MgCl₂ and 1 M levamisole. Reaction between alkaline phosphatase and the substrate gave a dark insoluble precipitate.

Secretion and Glycosylation of the Protein Encoded by pCTLA-ClfA

Further characterization of the expression of pCTLA-ClfA in a eukaryotic cells was performed by an immunoprecipitation assay. COS-1 cells were transfected as above described using 75 cm² culture flasks and 10 mM butyrate was added after 48 hours, with or without 3 μg/ml of tunicamycin to inhibit N-glycosylation. For each condition, the supernatant from a 75 cm2 culture flask was recovered 24 hours later and concentrated 10 fold using Centricon YM-30 (Millipore Canada Ltd., Nepean, Canada) in the presence of anti-proteases (2 μg/ml aprotinin, 2 mM EDTA and 100 μg/ml PMSF). The concentrated supernatant was incubated for 4 hours at 4° C. with 10 μl of rabbit anti-GST-ClfA(221-550). Controls were carried out using either preimmune rabbit serum with transfected cells or immune serum with non-transfected cells. Antigen-antibody complexes were precipitated by centrifugation of the supernatants after incubation with 50 μl of 10% protein-A Sepharose (Roche diagnostics) for 4 hours at 4° C. The pellet was repeatedly washed, resuspended in loading buffer and analysed by SDS-PAGE. The proteins were blotted onto Immobilon PVDF membrane (Millipore) with a semi-dry transfer apparatus (Bio-Rad Laboratories, Richmond, USA). A standard western blot was carried out using serum from mice immunized with plasmid pClfA (1/10 000), anti-mouse IgG HRP conjugate (1/30 000) and ECL chemiluminescence kit (Amersham Pharmacia Biotech).

DNA Immunization of Mice

The plasmids used for injection were purified using EndoFree Plasmid Giga Kit (Qiagen Inc., Mississauga, Canada) as specified by the manufacturer. The DNA concentration was evaluated by U.V absorbance at 260 nm and the plasmids diluted at 1 mg/ml in endotoxin-free phosphate buffered saline (PBS, Sigma). The vaccination experiments were conducted using eight groups of six BALB/c mice (14-16 gr; Charles River Laboratories Inc., St. Constant, Canada). Anaesthesia with ketamine/xylazine (87 and 13 mg/kg, respectively) was used to immobilize the mice for at least 15 minutes after each immunization. All the mice received injections at three weeks interval for a total of three immunizations (days 0, 21 and 42) with plasmid, plasmid and protein or protein alone. Immunizations were performed in the first 5 groups by using the following plasmids: pCI, pFnBPA, pCTLA-FnBPA, pClfA, pCTLA-ClfA. A single plasmid was used for each group. Mice were vaccinated intramuscularly (i.m.) with plasmid DNA as previously in the tibialis anterior muscle using two bilateral 50 μl injections, for a total of 100 μg of DNA, at each specified day. The recombinant protein ClfA(221-550) (25 μg) was used for the second boost (s.c.) in two additional groups where the first two injections were carried out using pClfA or pCTLA-ClfA. Finally, the last group was vaccinated with the ClfA(221-550) protein alone using the same schedule as indicated before. Sera were harvested at day 63 and kept at −20° C. The guidelines of the Canadian Council on Animal Care (CCAC) were respected during all the procedures (1993. Guide to the Care and Use of Experimental Animals. Olfert E. D., B. M. Cross and A. A. McWilliam (ed.). Vol 1. 2^{nd} edition. CACC, Ottawa, ON, Canada).

Antibody Assays

All the assays using antibodies from DNA vaccinated mice were performed with sera harvested on day 63, exactly 3 weeks after the last injection.

Total IgG: Enzyme-linked immunosorbant assays (ELISA) were used to determine the presence of IgG antibodies against adhesin antigens. Polystyrene Maxisorp 96-well plates (Nalge Nunc International Corp., Rochester, USA) were coated for 2 hours at 37° C. with either 50 μl of recombinant ClfA(221-550) or D1-D3 at a concentration of 10 mg/ml in carbonate/bicarbonate buffer at pH 9.6. Following saturation with powdered milk solution (5% w/v) overnight at 4° C., the diluted serum samples were added at the specified dilution and incubated for 2 hours at 37° C. A biotinylated anti-mouse IgG secondary antibody (1/1000) was added and incubated for 1 hour at 37° C. After incubation with streptavidin-HRP (Amersham Pharmacia Biotech) diluted 1/750, 100 μl of the chromogenic substrat solution (TetraMethylBenzene (TMB) 42 mM and 0.01% of hydrogen peroxide) was added. To stop the enzymatic reaction, 20 μl of H₂SO₄ 4N was used. Between each step, 3 washes with PBS-Tween 0.05% were carried out. The optical density (O.D.) was read on a plate reader at 450 nm (Bio-Tek Instruments, Winooski, USA). Each serum was tested individually in triplicate in two distinct experiments.

Isotypes: ELISA assays were carried out as before but the secondary antibody were either mouse anti-IgG1-HRP (1/500) or mouse anti-IgG2a-HRP (1/500) (BD Pharmingen Canada Inc., Mississauga, Canada) and the complex streptavidin-HRP addition was omitted. All the mouse serum samples were tested individually in triplicate at 1/750 dilution. The experiment was carried out twice with similar results.

Antibody binding to S. aureus: To detect the overall IgG binding of antibodies to bacteria, an ELISA was used with the bacteria as fixed antigen. A 1/10 dilution of an overnight culture of S. aureus strain Newman was applied to a 96 well plate in carbonate/bicarbonate buffer pH 9.6 and incubated overnight at 4° C. To reduce non-specific interactions with the mouse sample serum, preimmune rabbit serum (1/1000) was added for 1 hour 37° C. following the blocking step. The remaining steps were conducted as before using anti-mouse IgG-HRP conjugate as secondary antibody. This procedure was repeated twice.

Splenocyte Proliferation

At the specified day, the splenocytes of three mice from each vaccinated group were harvested, pooled and a single cell suspension was produced. Cells were distributed at 3×10⁵ cells per well of 96-well culture plate in RPMI 1640 containing 10% inactivated FBS, 10 mM HEPES, 2 mM glutamine, 1 mM pyruvate, 30 μM indomethacin, 5×10⁻⁵ M 2-mercaptoethanol and 50 μg/ml gentamicin. Different conditions of stimulation were used: 8 μg/ml of concanavalin A (con A) and medium alone were the controls whereas 2.5 μg of ClfA(221-550) was the test antigen. After stimulation for 72 hours at 37° C. with 5% CO₂, 1 μCi of [methyl-³H]-thymidine (specific activity: 5 Ci/ mmol, Amersham Pharmacia Biotech) was added to each well. Conditions used were determined in a preliminary experiment. Cells were harvested using a Skatron semi-automatic cell harvester (Molecular devices Corp., Sunnyvale, USA) and radioactivity incorporated after 14 hours of incubation was evaluated with a LS6000Sc beta counter (Beckman Coulter Canada Inc, Mississauga, Canada). The stimulation index (SI) was calculated as the CPM ratio of stimulated cells to non stimulated cells

Inhibition Assay of S. aureus Binding to Fibrinogen

The ability of antibodies produced by DNA vaccination against ClfA to inhibit binding of S. aureus to fibrinogen was evaluated using a test based on the adherence of radiolabelled bacteria. Labeling of bacteria was carried out using an overnight culture in tryptic soy broth (TSB). This culture was diluted 1/20 in 5 ml of TSB containing 1 mCi of [methyl-³H]-thymidine (specific activity: 5 Ci/ mmol, Amersham Pharmacia Biotech) and incubated for an additional 6 hours (37° C., 250 rpm). The bacteria were washed three times in PBS, resuspended in the initial volume and stored at −20° C. until use. For the inhibition assay, plates were coated for 2 hours at 37° C. with 1 μg of fibrinogen (Sigma) per well in 50 μl of carbonate/bicarbonate buffer pH 9.6. The remaining sites were blocked overnight by addition of skimmed milk powder in PBS at 5%. Labeled bacteria were preincubated for 1 hour with twofold serial dilutions of pooled sera (day 63) from the 6 mice immunized with either pCI or pClfA. The dilution of labeled bacteria used for the assay was chosen to give approximately 8000 CPM per well in the absence of inhibition. The bacteria were then added to the wells and incubated overnight at 4° C. After washing to remove non-adherent cells, the bacteria were detached by 2 incubations of 30 min with 100 μl of SDS (3%) and the radioactivity was counted in a beta counter. Each step was followed by 3 washes with PBS-Tween (0.01%). This assay was performed twice.

In vitro Opsonophagocytosis

Macrophages were recruited in BALB/c mice by intraperitoneal injection of 3% thioglycollate brewer medium and harvested 4 days later by peritoneal rinsing with 6 ml of cold PBS. The cells were washed three times, resuspended in DMEM containing 10% inactivated FBS and distributed in 24 well plates at 3×10⁵ cells per well. After 2 hours of incubation at 37° C. with 5% CO₂, the wells were rinsed twice with PBS to remove non adherent cells. Following a preincubation of S. aureus with sera (1/250 and 1/1000) from pCI or pClfA vaccinated mice as for the inhibition test, the bacteria were washed with PBS. These bacteria were then added (1.2×10⁷ bacteria in 300 μl) to the plate in quadruplicate for a ratio of bacteria to macrophages of approximately 40:1. Phagocytosis was stopped after 30 min by addition of cold PBS. Bacteria which bound to the macrophage without being phagocytosed were killed by incubation of the cells for 1 hour at 37° C. with DMEM in the presence of 50 μg/ml of gentamicin. After 3 washes with PBS, the macrophages were lysed for 20 minutes with sterile water. The efficiency of phagocytosis was estimated by measuring the number of live bacteria in the macrophage lysates. The number of CFU was evaluated after an overnight incubation at 37° C. of serial logarithmic dilutions of the lysates on tryptic soy agar plates. The two dilutions of sera analyzed in the assay were used in two separate experiments.

In vivo Opsonisation

The mice were vaccinated 3 times at three week intervals with 100 μg of plasmid ClfA or pCI and the serum was used three weeks after the last immunization. The bacteria (S. aureus Newman) were pre-incubated for 1 hour at 20 degrees with serum from the mice immunized with pClfA. These bacteria were injected into the mammary glands (1×10⁵ bacteria per gland) of mice. Five mice per group were used. After 20 hours the mammary glands were removed, homogenised and the number of colony forming units were measured on petri dishes. Using logarithmic dilutions.

Intraperitoneal Challenge of Mice Vaccinated with pClfA DNA

Mice vaccinated as described above were challenged with 0.75×10⁸ bacteria S. aureus Newman 2 weeks after the last injection. The bacteria were injected intraperitoneal (IP) in a volume of 1 ml of saline. After 5 days the livers were removed and CFU were counted after logarithmic dilution.

Statistics

The ELISA data were analyzed by ANOVA using the Bonferroni correction for multiples comparison when necessary. For other ELISA, opsonophagocytose, inhibition and splenocytes proliferation assays, the Student t-test was used.

### Results

Expression of Plasmid Constructions in COS-1 Cells

The ability of the plasmid vectors according to the invention pFnBP, pCTLA-FnBP, pClfA and pCTLA-ClfA to express their antigens was evaluated in COS-1 transfected cells. Since S. aureus adhesins originate from a prokaryotic organism, it was preferable to investigate expression in a eukaryotic environment. The antibodies used to detect expressed proteins in COS-1 cells were developed against recombinant bacterial expressed proteins. FIG. 2 shows darker cells expressing the bacterial adhesins binding domains. All the plasmids expressed the encoded proteins in COS-1 cells. Controls with preimmune serum were almost free of dark regions confirming the specificity of the immunohistochemistry technique for the bacterial antigens. Even though the pCTLA-FnBP and pCTLA-ClfA contained signal sequence for secretion, no quantitative differences can be seen on the pictures when compared to the non-secreted expressed antigens.

Secretion and Glycosylation of ClfA Encoded by Plasmids

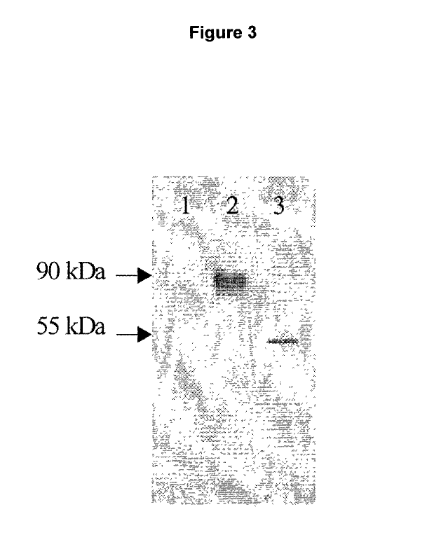

Transfections of COS-1 cells were also performed with pClfA and pCTLA-ClfA to confirm whether the protein CTLA-ClfA(221-550) was secreted and to determine if the ClfA portion of the protein was glycosylated in eukaryotic cells. Calculated molecular weight of CTLA-ClfA(221-550) was 55.5 kDa, the extracellular portion of CTLA is known to posses 2 N-glycosylation sites. Based on the amino acid sequence of ClfA(221-550), 8 other possible N-glycosylation sites were found. The results of the immunoprecipitation assays with the supernatants of transfected cells, in the presence or absence of tunicamycin, are presented in FIG. 3. In the absence of tunicamycin treatment, the band revealed by chemiluminesence gave an approximative weight of 90 kDa for CTLA-ClfA(221-550) (FIG. 3, lane 2). When the transfected cells where treated with tunicamycin, the apparent molecular weight was reduced to an estimated weight of 55 kDa (FIG. 3, lane 3). No band was visible for cells transfected with the non secreted form of antigen produced by the pClfA plasmid (FIG. 3, lane 1).

Humoral Immune Responses Induced by DNA Immunization

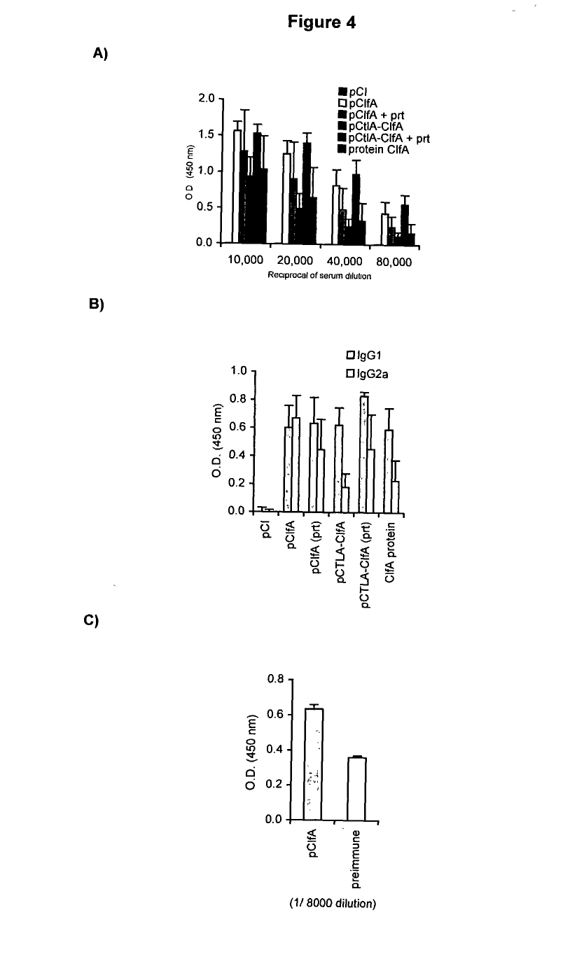

No signs of inflammation at the injection site and no mortality was observed in mice during the experiments. The vaccination with plasmids encoding the D1-D3 part of FnBP-A from S. aureus, pFnBPA and pCTLA-FnBPA, induced no measurable production of antibodies at 1/200 dilution in a biotin/streptavidin ELISA. In contrast, vaccination with pClfA and pCTLA-ClfA produced specific antibodies (FIG. 4A). The ELISA were carried out using recombinant antigen so the antibodies detected are those that recognized prokaryotic protein. Higher levels of antibodies were produced by immunizing with pClfA than with pCTLA-ClfA (FIG. 4A) (P<0.05). However, a second boost with 25 μg of recombinant ClfA(221-550) instead of 100 μg of DNA increased the level of antibodies of the animals immunized with pCTLA-ClfA (P<0.05) but not pClfA. Isotypes IgG1 and IgG2a were evaluated for each mouse as illustrated in FIG. 4B. When comparing the IgG isotypes within groups, only the groups vaccinated with the secreted antigen (pCTLA-ClfA) or the protein alone demonstrated a significative O.D. difference (IgG1>IgG2a, P<0.05). This indicates that immunization with pClfA, with or without a protein boost, induces a higher proportion of IgG2a in comparison with the other groups. Finally, recognition of native ClfA on the surface of S. aureus Newman by serum of pClfA vaccinated mice was evaluated by the ability of antibodies to bind to the bacteria. FIG. 4C shows the binding of IgG to the surface ClfA on S. aureus strain Newman in ELISA at a sera dilution of 1/8000. The eucaryote expressed bacterial antigen permitted the production of antibodies that recognized the whole bacteria when compared to preimmune serum. The ELISA results given in O.D. units represent the O.D. in the presence of antigen minus O.D. without antigen. Sera from mice immunized by pCI plasmid alone gave consistently the same background as preimmune sera.

Proliferation of Splenocytes

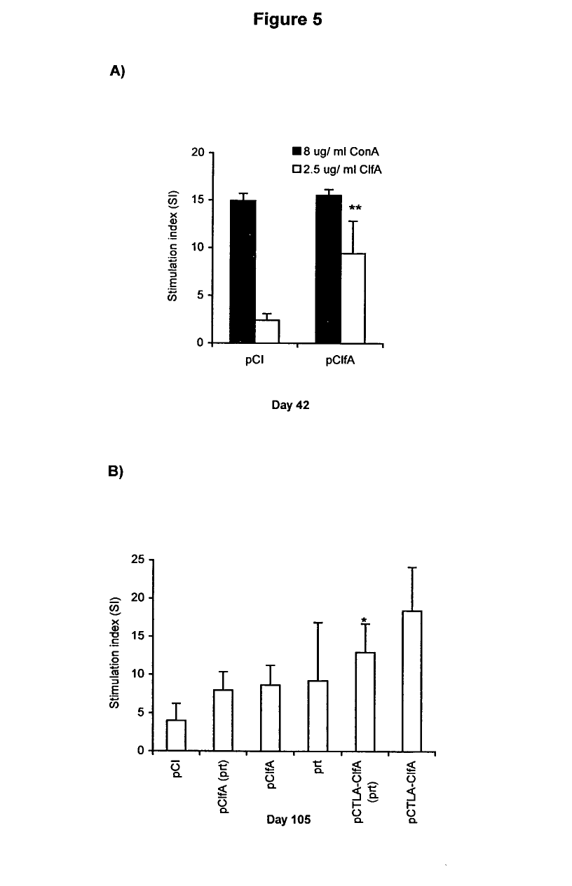

The implication of cells in the immune response was established by testing the capacity of splenocytes to proliferate in vitro in presence of the antigen. First, splenocytes from pClfA vaccinated mice were assayed 3 weeks after the second injection (day 42). The results shown in FIG. 5 demonstrate specific proliferation in presence of 2.5 μg/ml of recombinant ClfA(221-550) (P<0.01). In a subsequent experiment, all the vaccinated groups were assayed for splenocyte proliferation after a relatively long term period (day 105). In that case, only the splenocytes harvested from mice vaccinated with pCTLA-ClfA demonstrated a detectable proliferative response after stimulation with ClfA(221-550) (P<0.05 for pCTLA-ClfA and P<0.01 for pCTLA-ClfA+ClfA protein). Proliferation of spleenocytes from pCI vaccinated mice was considered as the baseline.

Functional Analysis of Antibodies from Sera of ClfA Vaccinated Mice

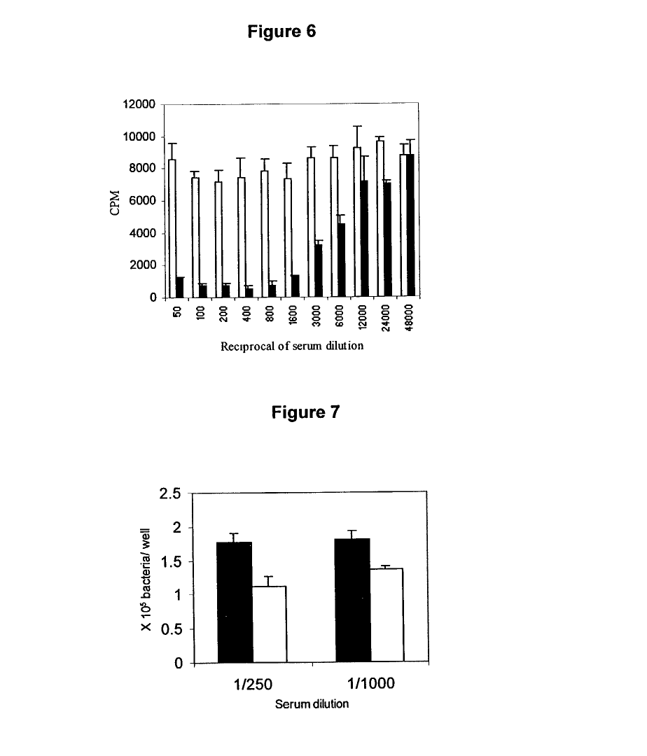

FIG. 6 shows the results of an experiment designed to determine the ability of antibodies against the binding portion of ClfA to block the interaction between bacteria and fibrinogen. After preincubation of bacteria, sera from mice vaccinated with pClfA inhibited binding of S. aureus to fibrinogen by up to 92%. This effect remained detectable at dilution 1/6000 (P<0.005). Moreover, as shown in FIG. 7, preincubation of S. aureus with serum from mice immunized with pClfA improved in vitro phagocytosis by mouse peritoneal induced macrophages. A serum dilution of 1/1000 increased the number of bacteria found within phagocytic cells by 32% as detected by counting viable bacteria on agar plates (P<0.01). When using a less diluted serum of 1/250, 60% more bacteria were ingested (P<0.001). In both assays, sera from mice vaccinated with the empty pCI plasmid vector constituted the control.

In vivo Opsonisation:

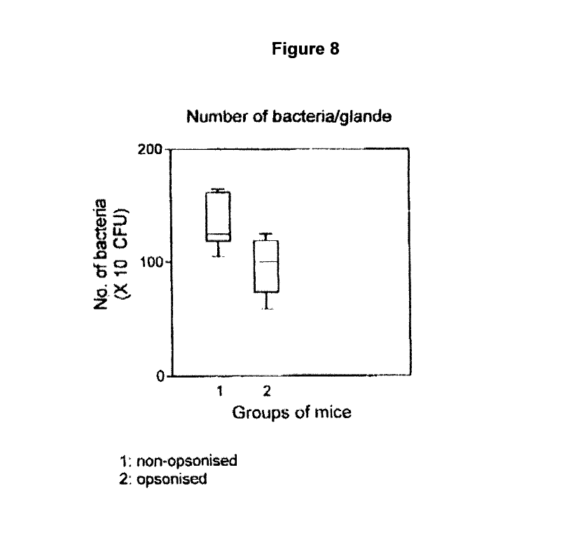

As shown in FIG. 8, there was a significant decrease in the number of bacteria in the glands that were infected with bacteria pre-treated with antibody.

Intraperitoneal Challenge of Mice Vaccinated with pClfA DNA

At a dilution of 10⁻⁵, no bacteria were detected in the mice vaccinated with ClfA (0/5) whereas in the mice vaccinated with pCI 3 out of 7 (3/7) were positive.

### Example 2

### DNA Vaccines According to the Present Invention Using Bicistronic Plasmid Vectors

In the present example, DNA vaccines of the present invention were performed in a mouse model. This ensured the proper function of the constructions in vivo, the amplitude and characteristics of the immune response, functional properties of antibodies.

### Methods

Animals: BALB/C mice of age 6-8 weeks old were separated in experimental groups of 16 mice for each group. Each experimental group were divided into three blocs of 4, 6 and 6 mice respectively to observe the immune response in a mastitis model.

2.2 Experimental Groups

The experimental groups were divided as follows.

| Group | Plasmid vector |
|---|---|
| 1 | pCl- extracellular part of Sortase |
| 2 | pCl-AIP |
| 3 | pCl-D1D3 of FnBp-A |
| 4 | pCl-Clfa |
| 5 | pCl-D1D3-Clfa |
| 6 | pCl-Clfa-extracellular part of Sortase |
| 7 | pCl-Clfa-AIP |
| 8 | pCl-Clfa-D1D3 |

Vaccination

Each mouse was injected with 100 μg DNA vaccine in the tibialis anterior muscle after anaesthesia with kétamine/xylazine in order to ensure mouse immobilization for about 30 minutes after the vaccination. There were three injections totally: one priming injection and two consecutive boosts at intervals of 21 days.

Sample Collection

Blood samples were collected every 10 days from the orbital plexus.

Determination of Titres of Anti-antigen Antibodies, (Total IgG), in Serum.

The level of anti-antigen antibodies, (total IgG), for each DNA vaccination were determined by ELISA using as a coating substrate the respective recombinant proteins. For preparing such ELISA, methods well known in the art may be used.

### Results

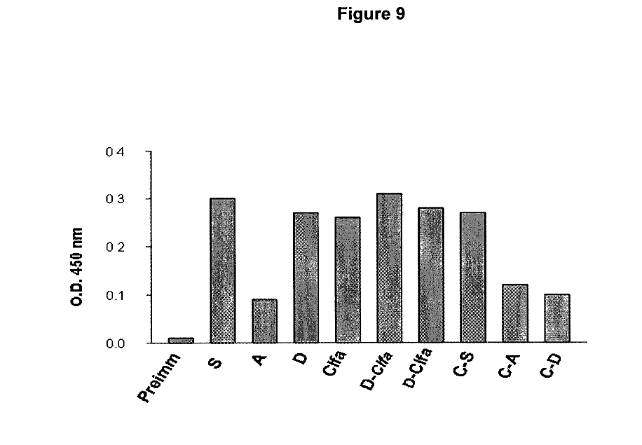

FIG. 9 shows the results of an ELISA test to mesure the antibody response to immunization with the plasmids containing individual and combinations of different S.aureus genes. The serum samples were diluted 1:250 in saline. The O.D. density of the pre-immune serum was arbitrarily given a value of 0.01 and deducted from the O.D. of the test sera. The letters represent the gene or combination of genes expressed by the plasmid. S=sortase, A=AIP, D=D1D3 of fibronectin binding protein, Clfa=Clumping factor A, D-Clfa=a single plasmid expressing both D1D3 and Clfa, but fused in the plasmid in the order D1D3 followed by Clfa, antibody against D1D3 was measured; the second D-Clfa is the same serum but tested against Clfa; C-S=a single plasmid expressing both Clfa and sortase, antibody against sortase; C-A=a single plasmid expressing both Clfa and AIP, antibody against AIP; C-D=a single plasmid expressing both ClfA and DID3 but in the order Clfa followed by D1D3, antibody measured against D1D3.

### Example 3

### DNA Vaccination of Dairy Cows Against S. aureus and Analysis of Their Immune Response

### Methods

Animals

Eight late gestation healthy heifers were used. During the vaccination period these animals were kept within the herd of the farm without any different treatment from the normal management conditions.

Experimental Groups

Eight heifers were divided in 2 groups. The first group of four heifers was vaccinated and the second group that was not vaccinated served as the control group.

Vaccination

Two months before calving heifers were primed with 2 mg of the DNA vaccine of the present invention. Heifers were boosted two times of intervals of 21 days with the same amount of DNA vaccine. This vaccination schedule provided the synchronization of the maximum magnitude of the expected immune response during the third or fourth week of lactation.

Immune Response Analysis in DNA Vaccinated Heifers

The overall immune response of DNA vaccinated heifers were analyzed through determination of humoral immune responses in serum and milk.

Humoral immunity was evaluated through ELISA determination in serum and milk of titres of antibodies anti-encoded antigens by DNA vaccine vectors for each of them separately, (total IgG).

### Results

Antibody Production by Cattle Against ClfA—DNA Plasmid Constructs

As shown in the following Table, administration of DNA vaccines according to the present invention elicited a humoral immune response in heifers. Because the ELISA used in the present example performed with an extremely low background, statistically significant OD values were observed. Indeed, by comparing the OD values of the control plasmid (pCI), it is clear that the heifers that received a DNA vaccine according to preferred embodiments of the present invention (see columns 2, 3 and 4) were provided with an immune response against proteins of S. aureus. This immune response was most evident two weeks after the second immunization. Some animals (appprox 30%) did not respond. Inclusion of GMCSF increased the response.

| | OD of ELISA IgG1 at 1:500 dilution two weeks | | | |
|---|---|---|---|---|
| | after each immunization | | | |
| | | | pCl-GMCSF | pCl-CTLA4- |
| Week | pCl | pCl-ClfA | +pCl-ClfA | −IgG-ClfA |
| Preimmune | 0.052 | 0.055 | 0.06 | 0.062 |
| (Average of | | | | |
| three weeks) | | | | |
| 6 | 0.055 | 0.051 | 0.052 | 0.07 |
| 9 | 0.052 | 0.075 | 0.200 | 0.11 |
| 12 | 0.054 | 0.075 | 0.062 | 0.080 |

Animals were immunized at week 4, 7, and 10.
SEM pCl = ±0.005, pCl-ClfA = ±0.003, pCl-GMCSF +pCl-ClfA = ±0.02 for day 9 , 0.003 for the other days.

Although preferred embodiments of the present invention have been described in detail herein and illustrated in the accompanying drawings, it is to be understood that the invention is not limited to these precise embodiments and that various changes and modifications may be effected therein without departing from the scope or spirit of the present invention.

What is claimed is:

1. A composition comprising a plasmid and a pharmaceutically acceptable carrier, said plasmid comprising:
at least one nucleotide coding sequence of a Staphylococcus aureus polypeptide selected from the group consisting of adhesion proteins, extracellular regulatory proteins, and autoinducing peptides; and
transcriptional and translational regulatory sequences operably linked to said nucleotide sequence for expressing said polypeptide in a mammal.

2. The composition of claim 1, wherein the adhesion protein is a protein selected from the group consisting of clumping factor A and fibronectin-binding protein A.

3. The composition of claim 1, wherein the adhesion protein consists of clumping factor A or a functional derivative thereof.

4. The composition of claim 3, wherein the nucleotide coding sequence comprises nucleotides 1 to 3499 of GenBank accession no. Z18852.

5. The composition of claim 3, wherein the nucleotide coding sequence comprises nucleotides 962 to 1951 of GenBank accession no. Z18852.

6. The composition of claim 1, wherein the adhesion protein consists of fibronectin-binding protein A or a functional derivative thereof.

7. The composition of claim 6, wherein the nucleotide coding sequence comprises nucleotides 1 to 3342 of GenBank accession no. J04151.

8. The composition of claim 6, wherein the nucleotide coding sequence comprises nucleotides 962 to 1951 of GenBank accession no. J04151.

9. The composition of claim 6, wherein the nucleotide coding sequence comprises nucleotides 2538 to 2578 of GenBank accession no. J04151.

10. The composition of claim 6, wherein the nucleotide coding sequence comprises nucleotides 962 to 1951 and nucleotides 2538 to 2578 of GenBank accession no. J04151

11. The composition of claim 1, wherein the extracellular regulatory protein consists of sortase-A or a functional derivative thereof.

12. The composition of claim 11, wherein the nucleotide coding sequence comprises nucleotides 1 to 1256 of GenBank accession no. AF162687.

13. The composition of claim 11, wherein the nucleotide coding sequence comprises nucleotides 443 to 1147 of GenBank accession no. AF162687.

14. The composition of claim 1, wherein the autoinducing proteins consists of a pre-pheromone or a functional derivative thereof.

15. The composition of claim 14, wherein the nucleotide coding sequence comprises nucleotides 1 to 1691 of GenBank accession no. AF026120.

16. The composition of claim 14, wherein the nucleotide coding sequence comprises nucleotides 158 to 180 of GenBank accession no. AF026120.

17. The composition of claim 2, wherein the plasmid comprises a nucleotide encoding sequence of the clumping factor A and a nucleotide encoding sequence of the fibronectin-binding protein A.

18. The composition of claim 2, wherein the plasmid comprises a nucleotide encoding sequence of the clumping factor A and a nucleotide encoding sequence of an extracellular regulatory protein.

19. The composition of claim 2, wherein the plasmid comprises a nucleotide encoding sequence of the clumping factor A and a nucleotide encoding sequence of an autoinducing protein.

20. The composition of claim 1, wherein the plasmid further comprises an antigen presenting cell targeting sequence.

21. The composition of claim 20, wherein the antigen presenting cell targeting sequence consists of a sequence encoding a cytotoxic lymphocyte T antigen 4.

22. A method for eliciting an immune response against Staphyloccocus aureus in a mammal, said method comprising the step of administrating to said mammal an effective amount of a composition as defined in claim 1.

23. The method of claim 22, wherein said immune response confers a protective immunity against mastitis.

24. A method of eliciting a protective immunity against a Staphyloccocus aureus associated disease in a mammal, said method comprising administering to said mammal an effective amount of a composition as defined in claim 1.

25. The method of claim 24, wherein the disease is selected from the group consisting of pneumonia, mastitis, phlebitis, meningitis, urinary tract infections, osteomyelitis and endocarditis.

26. The method of claim 25, wherein the disease is mastitis.

27. The method of claim 26, wherein the mammal consists of a human.

28. The method of claim 26, wherein the mammal consists of a bovine.

29. A DNA vaccine for preventing and/or treating a Staphyloccocus aureus associated disease, the vaccine comprising a plasmid and a pharmaceutically acceptable carrier, said plasmid comprising:
at least one nucleotide coding sequence of a Staphylococcus aureus polypeptide selected from the group consisting of adhesion proteins, extracellular regulatory proteins, and autoinducing peptides; and
transcriptional and translational regulatory sequences operably linked to said nucleotide sequence for expressing said polypeptide in a mammal.

30. A method for preventing and/or treating a Staphyloccocus aureus associated disease in a mammal, comprising the step of administering to said mammal an effective amount of a DNA vaccine as defined in claim 29.

31. The method of claim 30, wherein the disease is selected from the group consisting of pneumonia, mastitis, phlebitis, meningitis, urinary tract infections, osteomyelitis and endocarditis.

32. The method of claim 31, wherein the disease is mastitis.


SEQUENCE LISTING (10 sequences)

SEQ ID NO: 1 - DNA, 23 bp, artificial sequence
  Sequence completely synthesized
ccggatccgaaggtggccaaaat

SEQ ID NO: 2 - DNA, 27 bp, artificial sequence
  sequence completly synthesized
gctctagatcattcattttggccgctt

SEQ ID NO: 3 - DNA, 25 bp, artificial sequence
  sequence completly synthesized
ccggatccgtagctgcagatgcacc

SEQ ID NO: 4 - DNA, 29 bp, artificial sequence
  sequence completly synthesized
gctctagatcactcatcaggttgttcagg

SEQ ID NO: 5 - DNA, 27 bp, artificial sequence
  sequence completly synthesized
ggtctagaggacctcagcacatttgcc

SEQ ID NO: 6 - DNA, 21 bp, artificial sequence
  sequence completly synthesized
atccgggcatggttctggatc

SEQ ID NO: 7 - DNA, 14 bp, artificial sequence
  Sequence completely synthesized
tcgagccaccatgg

SEQ ID NO: 8 - DNA, 14 bp, artificial sequence
  Sequence completely synthesized
gatcccatggtggc

SEQ ID NO: 9 - DNA, 43 bp, artificial sequence
  Sequence completely synthesized
tctggtggcggtggctcgggcggaggtgggtcgggtggcggcg

SEQ ID NO: 10 - DNA, 47 bp, artificial sequence
  Sequence completely synthesized
gatccgccgccacccgacccacctccgcccgagccaccgccaccaga